(19) United States
(12) Patent Application Publication
(10) Pub. No.: US 2015/0359507 A1
(43) Pub. Date: Dec. 17, 2015

(54) ULTRASOUND DIAGNOSIS APPARATUS AND ULTRASOUND IMAGE PROCESSING METHOD

(71) Applicants: Kabushiki Kaisha Toshiba, Minato-ku (JP); Toshiba Medical Systems Corporation, Otawara-shi (JP)

(72) Inventors: Chihiro SHIBATA, Nasushiobara (JP); Tomohisa IMAMURA, Nasushiobara (JP); Masao TAKIMOTO, Otawara (JP); Shigemitsu NAKAYA, Nasushiobara (JP); Takuya SASAKI, Nasu (JP); Kuramitsu NISHIHARA, Otawara (JP); Mio AZEGAMI, Nasushiobara (JP); Yuko TAKADA, Otawara (JP)

(73) Assignees: Kabushiki Kaisha Toshiba, Minato-ku (JP); Toshiba Medical Systems Corporation, Otawara-shi (JP)

(21) Appl. No.: 14/835,048

(22) Filed: Aug. 25, 2015

Related U.S. Application Data
(63) Continuation of application No. PCT/JP2014/056505, filed on Mar. 12, 2014.

(30) Foreign Application Priority Data
Mar. 12, 2013 (JP) 2013-049745

(51) Int. Cl. A61B 8/06; A61B 8/08; A61B 8/14

(52) U.S. Cl. CPC A61B 8/06; A61B 8/14; A61B 8/5223; A61B 8/5207; A61B 8/5276

(57) ABSTRACT

According to one embodiment, an ultrasound diagnosis apparatus includes a tomographic image data generator, a blood flow information generator, a change detector, and a condition changing unit. The tomographic image data generator sequentially obtains tomographic image data of a subject for a plurality of times. The blood flow information generator performs processing on a reception signal based on signal processing conditions including a velocity threshold related to blood flow information of the subject to obtain the blood flow information. The change detector detects the magnitude of change in the time axis direction of a plurality of pieces of tomographic image data for different times. The condition changing unit changes the signal processing conditions based on the magnitude of change in the time axis direction. When the signal processing conditions are changed, the blood flow information generator performs the processing based on changed signal processing conditions.

### CROSS-REFERENCE TO RELATED APPLICATION

This application is based upon and claims the benefit of priority from Japanese Patent Application No. 2013-049745, filed 12/03/2013; the entire contents of which are incorporated herein by reference.

### FIELD

Embodiments described herein relate generally to an ultrasound diagnosis apparatus and an ultrasound image processing method.

### BACKGROUND

Ultrasound diagnosis apparatuses transmit ultrasound waves to a subject and receive reflected waves therefrom via an ultrasound probe to thereby acquire biological information of the subject.

Some ultrasound diagnosis apparatuses generate a blood flow image (color flow mapping (CFM) image) together with a tomographic image (B-mode image) at regular intervals to generate moving image information.

The blood flow image is generated based on a Doppler signal. In the blood flow image, blood flow information obtained based on the Doppler signal is associated with color information, and a two-dimensional blood flow image is displayed according to the color information. Thus, a blood flow is visualized in the blood flow image.

Doppler signals may sometimes contain clutter components induced by the movement of subject tissue such as beating and peristalsis of an organ. When the subject tissue makes large movement during ultrasound diagnosis, Doppler signals contain many clutter components, and when it makes less movement, the signals contain less clutter components. If the clutter contained in the Doppler signal is directly visualized in a blood flow image, the clutter is rendered in a partial region of the image. Since the image of the clutter rendered in the blood flow image does not represent blood flow, it may lead to misdiagnosis. Besides, Doppler signals may sometimes contain motion artifact generated by the movement of the ultrasound probe. The movement of the ultrasound probe can be caused by, for example, the hand movement of the user. When the ultrasound probe makes large movement during ultrasound diagnosis, Doppler signals contain many motion artifacts, and when it makes less movement, the signals contain less motion artifacts. If the motion artifact contained in the Doppler signal is directly visualized in a blood flow image, the motion artifact is rendered in a wide region of the image. The image of such motion artifact poses an obstacle to the user in visually checking a tomographic image and an image representing a blood flow. In other words, the image of the motion artifact is obtrusive to the user. For these reasons, there is a demand to reduce clutter and motion artifacts for the ultrasound diagnosis apparatuses.

### BRIEF DESCRIPTION OF THE DRAWINGS

FIG. 1 is a block diagram of an ultrasound diagnosis apparatus according to an embodiment;

FIG. 2 is a block diagram of the ultrasound diagnosis apparatus of the embodiment;

FIG. 3 is a table for explaining the outline of the ultrasound diagnosis apparatus of the embodiment;

FIG. 4 is a flowchart of the operation of the ultrasound diagnosis apparatus in the embodiment;

FIG. 5A is an example of an ultrasound image generated by the ultrasound diagnosis apparatus of the embodiment;

FIG. 5B is an image for comparison with the ultrasound image generated by the ultrasound diagnosis apparatus of the embodiment;

FIG. 6 is a block diagram of an ultrasound diagnosis apparatus according to a modification of the embodiment;

FIG. 7 is a flowchart of the operation of the ultrasound diagnosis apparatus in the modification; and

FIG. 8 is a block diagram of an ultrasound diagnosis apparatus according to another modification of the embodiment.

### DETAILED DESCRIPTION

In general, according to one embodiment, an ultrasound diagnosis apparatus includes a tomographic image data generator, a blood flow information generator, a change detector, and a condition changing unit. The tomographic image data generator receives a reception signal based on reflected waves returned from a subject in response to ultrasound waves transmitted to the subject, and sequentially obtains tomographic image data of the subject for a plurality of times. The blood flow information generator performs processing on the reception signal based on signal processing conditions including a velocity threshold related to blood flow information of the subject to obtain the blood flow information. The change detector receives a plurality of pieces of tomographic image data for different times, and detects the magnitude of change in the time axis direction of the data. The condition changing unit changes the signal processing conditions based on the magnitude of change in the time axis direction detected by the change detector. When condition changing unit changes the signal processing conditions, the blood flow information generator performs the processing based on changed signal processing conditions to obtain the blood flow information.

According to another embodiment, an ultrasound image processing method is directed to the processing of a reception signal based on reflected waves returned from a subject in response to ultrasound waves transmitted to the subject. The ultrasound image processing method includes: sequentially obtaining tomographic image data of the subject for a plurality of times; performing processing on the reception signal based on signal processing conditions including a velocity threshold related to blood flow information of the subject to obtain the blood flow information; detecting the magnitude of change in the time axis direction of a plurality of pieces of tomographic image data for different times; and, when having changed the signal processing conditions based on the magnitude of change in the time axis direction, obtaining the blood flow information based on changed signal processing conditions.

### [Configuration]

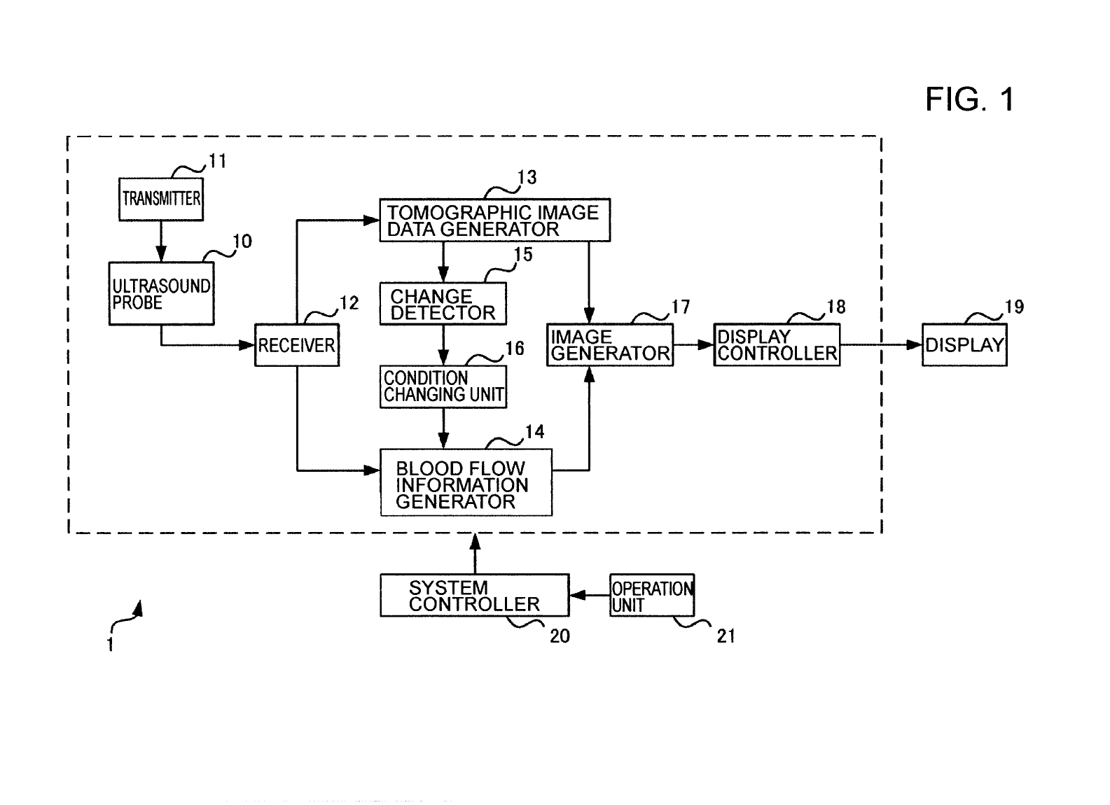

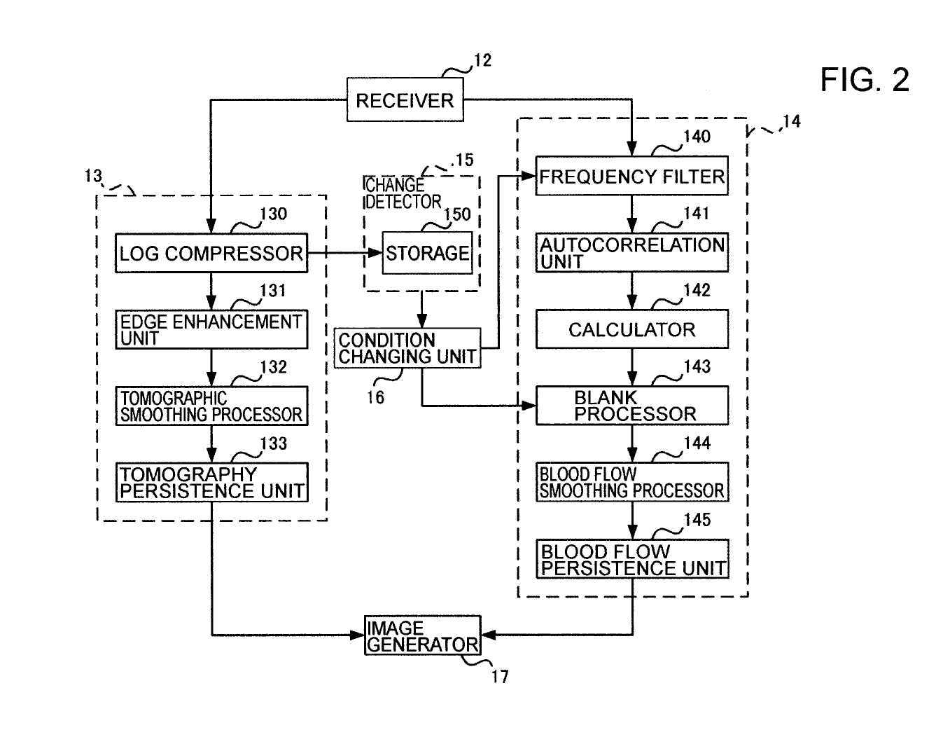

FIGS. 1 and 2 are block diagrams illustrating a configuration of an ultrasound diagnosis apparatus 1 according to this embodiment. The ultrasound diagnosis apparatus 1 includes an ultrasound probe 10, a transmitter 11, a receiver 12, a tomographic image data generator 13, a blood flow information generator 14, a change detector 15, a condition changing unit 16, an image generator 17, a display controller 18, a display 19, a system controller 20, and an operation unit 21.

### (Ultrasound Probe 10)

The ultrasound probe 10 may be a one-dimensional array probe including an array of a plurality of ultrasound transducers arranged in a scanning direction, or a two- dimensional array probe including two-dimensional arrays of a plurality of ultrasound transducers. The ultrasound probe 10 may also be a mechanical one-dimensional array probe formed of a plurality of ultrasound transducers that are arranged in an array in the scanning direction and are swung in a swing direction perpendicular to the scanning direction. The ultrasound probe 10 transmits ultrasound waves to a subject, and receives reflected waves therefrom as an echo signal.

### (Transmitter 11)

The transmitter 11 supplies electrical signals to the ultrasound probe 10 to generate ultrasound waves. The transmitter 11 includes a transmission delay circuit and a pulsar circuit (not illustrated). The transmission delay circuit adds delay to the transmission of ultrasound waves to implement transmission focus. The pulser circuit includes as many pulsers as paths (channels) corresponding to the ultrasound transducers. The pulser circuit generates a driving pulse to be supplied to the ultrasound transducers of the ultrasound probe 10 at the transmission timing delayed.

### (Receiver 12)

The receiver 12 is fed with an echo signal received by the ultrasound probe 10. Upon receipt of the echo signal, the receiver 12 adds delay to the echo signal to convert the analog echo signal to digital data having been subjected to phasing (i.e., subjected to beam forming).

The receiver 12 includes, for example, a preamplifier circuit, an A/D converter, a reception delay circuit, and an adder (not illustrated). The preamplifier circuit amplifies echo signals output from the ultrasound transducers of the ultrasound probe 10 with respect to each receiver channel. The A/D converter converts the amplified echo signals to digital signals. The reception delay circuit assigns the echo signals each converted into a digital signal with a delay time required to determine the reception directivity. The adder performs phasing and adding of the echo signals having the delay time. Thereby, a reflection component from a direction corresponding to the reception directivity is emphasized. The receiver 12 performs quadrature detection on a signal obtained by the addition, and outputs it as a reception signal to the tomographic image data generator 13 and the blood flow information generator 14.

### (Tomographic Image Data Generator 13)

The tomographic image data generator 13 receives the reception signal from the receiver 12. By sending ultrasound waves to a subject, the reception signal is obtained based on reflected waves therefrom. The tomographic image data generator 13 sequentially obtains tomographic image data of the subject for a plurality of times. In other words, based on the reception signal received from the receiver 12, the tomographic image data generator 13 obtains the tomographic image data at regular intervals (at a predetermined frame rate). The tomographic image data obtained with respect to a certain time corresponds to a still image at the time (frame). The tomographic image data includes output from any of a log compressor 130, an edge enhancement unit 131, a tomographic smoothing processor 132, and a tomography persistence unit 133 (described later). The tomographic image data generator 13 includes the log compressor 130, the edge enhancement unit 131, the tomographic smoothing processor 132, and the tomography persistence unit 133. The log compressor 130 compresses the reception signal by logarithmic conversion, and outputs it to the edge enhancement unit 131 and a storage 150. The edge enhancement unit 131 enhances the edges of the signal received from the log compressor 130, and outputs it to the tomographic smoothing processor 132. The tomographic smoothing processor 132 performs filtering to smooth the signal received from the edge enhancement unit 131, and outputs it to the tomography persistence unit 133. The tomography persistence unit 133 performs weighted addition of the signal newly received from the tomographic smoothing processor 132 to signals received therefrom in the past. The tomography persistence unit 133 outputs the signal obtained by the weighted addition to the image generator 17.

### (Blood Flow Information Generator 14)

The blood flow information generator 14 receives the reception signal from the receiver 12, and performs processing based on specified signal processing conditions on the reception signal to obtain blood flow information of the subject. Having received the reception signal, the blood flow information generator 14 obtains the blood flow information of the subject by performing processing on the reception signal based on the signal processing conditions including a velocity threshold related to the blood flow information. The signal processing conditions include frequency characteristics, a velocity threshold, a variance threshold, a lower power threshold, or an upper power threshold, or a combination of these (described later). The blood flow information includes the velocity, variance, or power of the blood flow in the subject, or a combination of these. The blood flow information generator 14 obtains the blood flow information at regular intervals. The blood flow information obtained with respect to a certain time corresponds to a still image at the time (frame). When the condition changing unit 16 changes the signal processing conditions, the blood flow information generator 14 performs processing on the reception signal based on changed signal processing conditions to obtain the blood flow information. The signal processing conditions may be specified by the user, or may be preset. The blood flow information generator 14 includes a frequency filter 140, an autocorrelation unit 141, a calculator 142, a blank processor 143, a blood flow smoothing processor 144, and a blood flow persistence unit 145.

The frequency filter 140 performs filtering on the reception signal received from the receiver 12 based on specified frequency characteristics. When the condition changing unit 16 changes the frequency characteristics, the frequency filter 140 performs filtering on the reception signal based on changed frequency characteristics. Thereby, the frequency filter 140 reduces clutter in the reception signal. Examples of the frequency characteristic include a filter type such as Butterworth, Bessel, and Chebyshev, cut-off frequency of each filter type, a passband, and a filter order. The frequency filter 140 outputs the signal subjected to the filtering to the autocorrelation unit 141.

The autocorrelation unit 141 performs an autocorrelation calculation with respect to the signal received from the frequency filter 140. The autocorrelation unit 141 outputs the signal, on which the autocorrelation calculation has been performed, to the calculator 142.

The calculator 142 receives the signal from the autocorrelation unit 141, and calculates the velocity of blood flow in the subject. The calculator 142 outputs the velocity calculated to the blank processor 143. In addition, the calculator 142 calculates the variance of blood flow in the subject. The calculator 142 outputs the variance calculated to the blank processor 143. Further, the calculator 142 calculates the power of blood flow in the subject. The calculator 142 outputs the power calculated to the blank processor 143.

The blank processor 143 stores a velocity threshold to obtain the velocity calculated by the calculator 142 as blood flow information. Of velocities received from the calculator 142, the blank processor 143 outputs a velocity equal to or above the velocity threshold to the blood flow smoothing processor 144. That is, from velocities received from the calculator 142, the blank processor 143 removes a velocity less than the velocity threshold as has been calculated from clutter, and outputs the rest to the blood flow smoothing processor 144. Incidentally, when the condition changing unit 16 changes the velocity threshold, the blank processor 143 outputs a velocity equal to or above a new velocity threshold to the blood flow smoothing processor 144.

The blank processor 143 also stores a variance threshold to obtain the variance calculated by the calculator 142 as blood flow information. Of variances received from the calculator 142, the blank processor 143 outputs a variance equal to or above the variance threshold to the blood flow smoothing processor 144. That is, from variances received from the calculator 142, the blank processor 143 removes a variance less than the variance threshold as has been calculated from clutter, and outputs the rest to the blood flow smoothing processor 144. Incidentally, when the condition changing unit 16 changes the variance threshold, the blank processor 143 outputs a variance equal to or above a new variance threshold to the blood flow smoothing processor 144.

Further, the blank processor 143 stores a lower power threshold and an upper power threshold to obtain the power calculated by the calculator 142 as blood flow information. Of powers received from the calculator 142, the blank processor 143 outputs a power equal to or above the lower power threshold as well as equal to or below the upper power threshold to the blood flow smoothing processor 144. That is, from powers received from the calculator 142, the blank processor 143 removes a power below the lower power threshold and above the upper power threshold as has been calculated from clutter, and outputs the rest to the blood flow smoothing processor 144. Incidentally, when the condition changing unit 16 changes the lower power threshold and the upper power threshold, the blank processor 143 outputs a power equal to or above a new lower power threshold as well as equal to or below a new upper power threshold to the blood flow smoothing processor 144.

Upon receipt of the output from the blank processor 143, the blood flow smoothing processor 144 performs smoothing filtering thereon, and outputs it to the blood flow persistence unit 145. In this embodiment, although an example is described in which the blank processor 143 receives output from the calculator 142, and the blood flow smoothing processor 144 receives output from the blank processor 143, this is not so limited. The blood flow smoothing processor 144 may receive output from the calculator 142 and output from the blank processor 143.

The blood flow persistence unit 145 performs weighted addition of a signal newly received from the blood flow smoothing processor 144 to signals received therefrom in the past. The blood flow persistence unit 145 outputs the signal obtained by the weighted addition to the image generator 17.

### (Change Detector 15)

The change detector 15 receives a plurality of pieces of tomographic image data for different times from the tomographic image data generator 13, and detects the magnitude of change in the time axis direction of the pieces of tomographic image data. The change detector 15 retrieves, from the storage 150, a latest frame corresponding to tomographic image data obtained as to the most recent time, and a previous frame corresponding to tomographic image data obtained as to the time before the most recent time. The change detector 15 calculates the average of signal amplitudes in the latest frame as a latest average value, and the average of signal amplitudes in the previous frame as a previous average value. The change detector 15 calculates a difference between the latest average value and the previous average value, and determines the difference as the magnitude of change in the time axis direction of tomographic image data. In this case, the larger the difference is, the larger magnitude of change is detected. The change detector 15 outputs the magnitude of change thus detected to the condition changing unit 16. Incidentally, when the storage 150 does not store the previous frame, the change detector 15 does not detect the magnitude of change in the time axis direction of tomographic image data.

Described below is a relationship between the change of the tomographic image data in the time axis direction and the movement of subject tissue as well as the movement of the ultrasound probe 10. The change of the tomographic image data in the time axis direction refers to the difference in the shape of the subject tissue (contents of the image) rendered in the previous frame and the latest frame. For example, when clutter occurs due to the movement of the subject tissue, a difference is caused in a partial image corresponding to the tissue having moved. Besides, when a motion artifact is generated due to the movement of the ultrasound probe 10, the tissue is rendered in an image as having moved in parallel or rotated. If the subject tissue or the ultrasound probe 10 moves while the ultrasound diagnosis apparatus 1 is capturing tomographic images at a predetermined frame rate, a difference is caused in the shape of the subject tissue rendered (contents of the image) between the previous frame and the latest frame. The larger movement of the subject tissue or the ultrasound probe 10 causes a greater change in the shape of the subject tissue rendered in the tomographic images. The change detector 15 detects the magnitude of the change, and outputs it to the condition changing unit 16.

The change detector 15 may divide the latest frame and the previous frame into a plurality of regions and calculate a latest average value and a previous average value to obtain the difference therebetween with respect to each region to thereby detect the magnitude of change in the time axis direction of tomographic image data.

The change detector 15 may also calculate the average value of signals of the latest frame and the average value of signals of the previous frame with respect to a designated partial region (region of interest), and obtain the difference between the average values to thereby detect the magnitude of change in the time axis direction of tomographic image data. In this case, the partial region may be specified by the user, or may be preset.

Further, the change detector 15 may obtain the similarity between the latest frame and the previous frame by cross-correlation analysis, and use the similarity as the magnitude of change in the time axis direction of tomographic image data. In this case, the lower the similarity is, the greater the detected change is.

Still further, the change detector 15 may retrieve the latest frame and a plurality of previous frames of different times from the storage 150. In this case, the change detector 15 detects the magnitude of change in the time axis direction of tomographic image data based on three or more frames. For example, the change detector 15 obtains the magnitude of change in the time axis direction of previous tomographic image data from previous frames of two times, and also obtains the magnitude of change in the time axis direction of the latest tomographic image data from the latest frame and a previous frame of a time closest to the latest frame. The change detector 15 adds weight to the magnitude of change in the time axis direction of the previous tomographic image data, and adds it to the magnitude of change in the time axis direction of the latest tomographic image data or multiplying the magnitude of change by it to detect the magnitude of change in the time axis direction of tomographic image data. Besides, for example, the change detector 15 extrapolates a plurality of signals of the previous frame and predicts a frame of the same time as the latest frame. The frame predicted is referred to as “predicted frame”. The change detector 15 calculates a difference between the predicted frame and the latest frame retrieved from the storage 150. The change detector 15 adds weight to the difference, and adds it to the magnitude of change in the time axis direction of the latest tomographic image data or multiplying the magnitude of change by it to detect the magnitude of change in the time axis direction of tomographic image data.

The change detector 15 includes the storage 150. The storage 150 stores the tomographic image data received from the log compressor 130. Here, the storage 150 stores at least tomographic image data of the number of frames that the change detector 15 uses for detecting the magnitude of change in the time axis direction of tomographic image data. For example, when the change detector 15 detects the magnitude of change in the time axis direction of tomographic image data based on the latest frame and one previous frame, the storage 150 stores two pieces of tomographic image data for the latest frame and the one previous frame. When the change detector 15 detects the magnitude of change in the time axis direction of tomographic image data, the storage 150 deletes the one previous frame stored therein, and a frame stored as the latest frame becomes a previous frame. Then, the storage 150 stores new tomographic image data as the latest frame. Similarly, when the change detector 15 detects the magnitude of change in the time axis direction of tomographic image data based on n frames, the storage 150 stores the latest frame and n-1 previous frames. When the change detector 15 detects the magnitude of change in the time axis direction of tomographic image data, the storage 150 deletes the oldest one of the n-1 previous frames, and stores new tomographic image data as the latest frame. In addition, when there is freeze operation or operation of changing the transmission conditions of ultrasound waves during ultrasound diagnosis, the storage 150 may delete all tomographic image data stored in response to a corresponding control signal from the system controller 20. The storage 150 may receive the output to be stored from the receiver 12, the edge enhancement unit 131, the tomographic smoothing processor 132, or the tomography persistence unit 133, instead of from the log compressor 130.

### (Condition Changing Unit 16)

The condition changing unit 16 changes the signal processing conditions based on the magnitude of change in the time axis direction of tomographic image data detected by the change detector 15. For example, among the signal processing conditions, the condition changing unit 16 changes the frequency characteristic of the frequency filter 140. The condition changing unit 16 stores, in advance, the magnitude of change in the time axis direction of tomographic image data in association with the frequency characteristics. The condition changing unit 16 retrieves the frequency characteristics associated with the magnitude of change in the time axis direction of tomographic image data detected by the change detector 15, and changes the frequency characteristics of the frequency filter 140 to the retrieved characteristics.

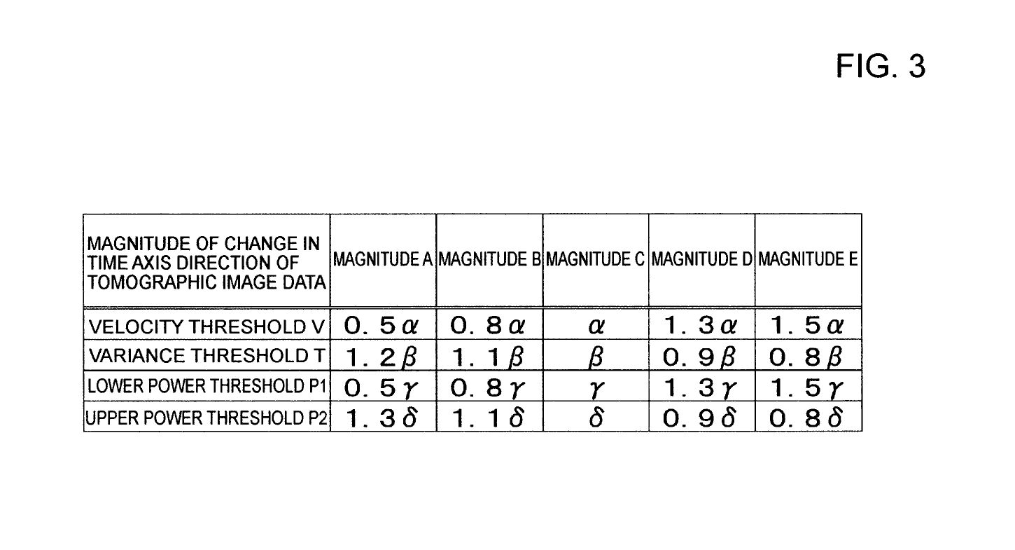

The condition changing unit 16 may change, for example, the velocity threshold of the signal processing conditions. In this case, the greater the change the change detector 15 has detected in the time axis direction of tomographic image data, the condition changing unit 16 raises the velocity threshold. The condition changing unit 16 may change, for example, the variance threshold of the signal processing conditions. In this case, the greater the change the change detector 15 has detected in the time axis direction of tomographic image data, the condition changing unit 16 reduces the variance threshold. The condition changing unit 16 may change, for example, the lower power threshold and the upper power threshold of the signal processing conditions. In this case, the greater the change the change detector 15 has detected in the time axis direction of tomographic image data, the condition changing unit 16 increases the lower power threshold and lowers the upper power threshold. For example, the condition changing unit 16 stores the magnitude of change in the time axis direction of tomographic image data in association with the velocity threshold, the variance threshold, the lower power threshold, and the upper power threshold. The condition changing unit 16 retrieves the velocity threshold, the variance threshold, the lower power threshold, and the upper power threshold associated with the magnitude of change in the time axis direction of tomographic image data received from the change detector 15, and outputs these thresholds to the blank processor 143 to change them in the blank processor 143. FIG. 3 illustrates an example of the magnitude of change in the time axis direction of tomographic image data, and the velocity threshold V, the variance threshold T, the lower power threshold P1 and the upper power threshold P2 associated therewith, that the condition changing unit 16 stores. In FIG. 3, the magnitude of change in the time axis direction of tomographic image data includes magnitude A, magnitude B, magnitude C, magnitude D, and magnitude E, which are greater in this order. The magnitude C is associated with the value “α” of the velocity threshold V, the value “β” of the variance threshold T, the value “γ” of the lower power threshold P1, and the value “δ” of the upper power threshold P2. Other magnitudes of change in the time axis direction of tomographic image data (the magnitude A, the magnitude B, the magnitude D, the magnitude E) are associated with values obtained by multiplying the values “α”, “β”, “γ”, and “δ” by coefficients illustrated in FIG. 3. The values may be specified by the user as, for example, “α”=0.1, “β”=0.9, “γ”=0.8, and “δ”=0.1, or may be automatically preset.

### (Image Generator 17)

The image generator 17 generates ultrasound image data based on the tomographic image data output by the tomographic image data generator 13 and the blood flow information output by the blood flow information generator 14. The image generator 17 includes, for example, a digital scan converter (DSC). The image generator 17 converts the blood flow information and the tomographic image data represented by signal strings of scan lines into image data represented by the orthogonal coordinate system (scan conversion). The ultrasound image data generated by the image generator 17 represents an image composed of a tomographic image (B-mode image) representing the tomographic image data (B-mode image data) and a blood flow image (color Doppler image) representing the blood flow information (color Doppler information), which are superimposed one on top of the other. The image generator 17 outputs the ultrasound image data generated to the display controller 18.

### (Display Controller 18)

Upon receipt of the ultrasound image data from the image generator 17, the display controller 18 displays an ultrasound image on the display 19 based on the ultrasound image data.

The display 19 displays an ultrasound image. The display 19 is formed of a display device such as, for example, a cathode ray tube (CRT) or a liquid crystal display (LCD). The display 19 need not necessarily be integrated with the ultrasound diagnosis apparatus 1. The display 19 may be configured to display an ultrasound image as being controlled by the display controller 18 via a common interface.

### (System Controller 20)

The system controller 20 controls each unit of the ultrasound diagnosis apparatus 1. The system controller 20 includes, for example, a storage device and a processor. The storage device stores computer programs to implement the functions of each unit of the ultrasound diagnosis apparatus 1. The processor executes the computer programs to implement the above functions.

### (Operation Unit 21)

The operation unit 21 is operated by the user and feeds each unit of the apparatus with signals or information corresponding to the contents of the operation. The operation unit 21 includes, for example, a keyboard, a mouse, a touch panel, and the like. The operation unit 21 need not necessarily be integrated with the ultrasound diagnosis apparatus 1. The operation unit 21 may be configured to feed signals and information to each unit via a common interface.

### [Operation]

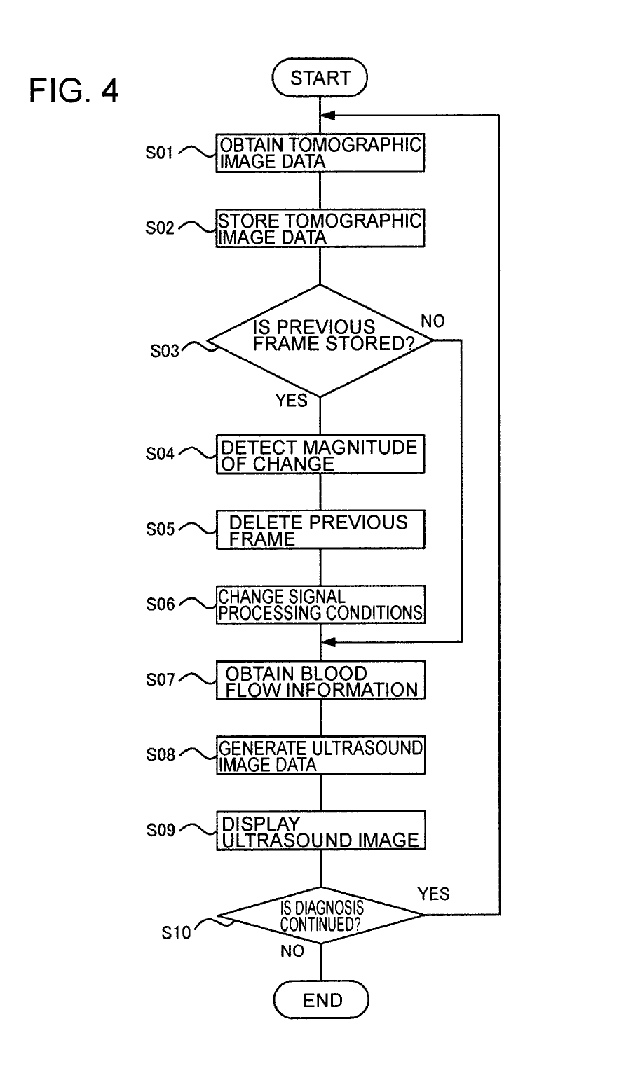

FIG. 4 is a flowchart illustrating the operation of the ultrasound diagnosis apparatus 1 of this embodiment.

### (S01)

In response to ultrasound waves transmitted to a subject, the tomographic image data generator 13 receives, from the receiver 12, a reception signal based on reflected waves from the subject, and obtains tomographic image data of the subject. At this time, the log compressor 130 performs compression by logarithmic conversion on the reception signal, and outputs it to the edge enhancement unit 131 and the storage 150. The edge enhancement unit 131 emphasizes the edges of the signal received from the log compressor 130, and outputs it to the tomographic smoothing processor 132. The tomographic smoothing processor 132 performs smoothing filtering on the signal received from the edge enhancement unit 131, and outputs it to the tomography persistence unit 133. The tomography persistence unit 133 performs weighted addition of the signal newly received from the tomographic smoothing processor 132 to signals received therefrom in the past. The tomography persistence unit 133 outputs the resultant signal to the image generator 17.

### (S02)

The storage 150 stores the tomographic image data received from the log compressor 130.

### (S03)

Besides the latest tomographic image data (latest frame) received from the log compressor 130, if the storage 150 stores previous tomographic image data (previous frame) obtained previous to the latest data (YES in step S03), the process proceeds to step S04. When the storage 150 does not store the previous tomographic image data (previous frame) as well as the latest tomographic image data (latest frame) received from the log compressor 130 (NO in step S03), the process proceeds to step S07.

### (S04)

The change detector 15 retrieves, from the storage 150, the latest frame as tomographic image data obtained as to the most recent time, and a previous frame as previous tomographic image data obtained as to the time before the most recent time. The change detector 15 detects the magnitude of change in the time axis direction of tomographic image data based on the tomographic image data retrieved. The change detector 15 outputs the magnitude of change thus detected to the condition changing unit 16.

### (S05)

The storage 150 deletes the previous frame stored therein, and a frame stored as the latest frame becomes a previous frame.

### (S06)

The condition changing unit 16 changes the signal processing conditions of the blood flow information generator 14 based on the magnitude of change in the time axis direction of tomographic image data detected by the change detector 15. The signal processing conditions includes the frequency characteristics of the frequency filter 140, the velocity threshold V, the variance threshold T, or the lower power threshold P1 and the upper power threshold P2 of the blank processor 143, or the combination thereof. In this case, the greater the change the change detector 15 has detected in the time axis direction of tomographic image data, the condition changing unit 16 raises the velocity threshold V, reduces the variance threshold T, increases the lower power threshold P1, and lowers the upper power threshold P2.

### (S07)

Upon receipt of the reception signal from the receiver 12, the blood flow information generator 14 performs processing on the reception signal based on the signal processing conditions to obtain blood flow information of the subject. At this time, the frequency filter 140 performs filtering based on the frequency characteristics on the reception signal received from the receiver 12. The autocorrelation unit 141 performs an autocorrelation calculation with respect to the signal received from the frequency filter 140. The calculator 142 calculates the velocity, variance, or power of the blood flow in the subject, or a combination of these. Of velocities received from the calculator 142, the blank processor 143 outputs a velocity equal to or above the velocity threshold to the blood flow smoothing processor 144. Further, of variances received from the calculator 142, the blank processor 143 outputs a variance equal to or above the variance threshold to the blood flow smoothing processor 144. Still further, of powers received from the calculator 142, the blank processor 143 outputs a power equal to or above the lower power threshold as well as equal to or below the upper power threshold to the blood flow smoothing processor 144. The blood flow smoothing processor 144 performs smoothing filtering on the output from the blank processor 143, and outputs it to the blood flow persistence unit 145. The blood flow persistence unit 145 performs weighted addition of the signal newly received from the blood flow smoothing processor 144 to signals received therefrom in the past. The blood flow persistence unit 145 outputs the signal obtained by the weighted addition to the image generator 17.

### (S08)

The image generator 17 generates ultrasound image data based on the tomographic image data output from the tomographic image data generator 13 and the blood flow information output from the blood flow information generator 14.

### (S09)

The display controller 18 receives the ultrasound image data from the image generator 17, and displays an ultrasound image on the display 19 based on the ultrasound image data.

### (S10)

When the ultrasound diagnosis is continued (YES in step S10), the process returns to the step S01. If not (NO in step S10), the operation ends.

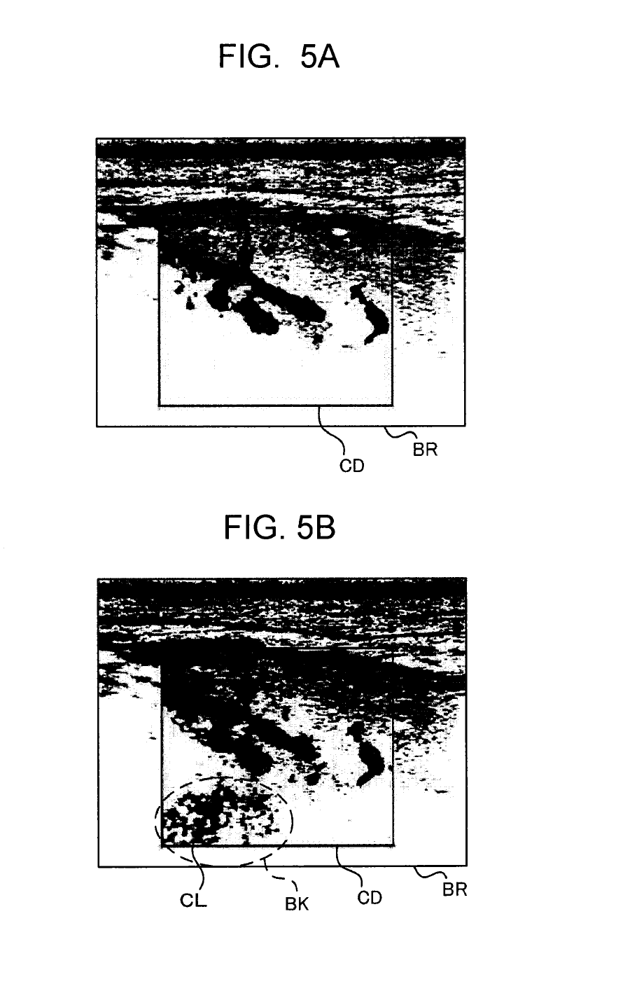

FIG. 5A illustrates an ultrasound image displayed by the ultrasound diagnosis apparatus 1 of the embodiment. FIG. 5B illustrates an ultrasound image without the functions of the change detector 15 and the condition changing unit 16 of this embodiment. For purposes of illustration, black and white are inverted in FIGS. 5A and 5B. In FIGS. 5A and 5B, a tomographic image BR and a blood flow image CD are superimposed one on top of the other. In FIG. 5B, an image CL of clutter is rendered in a region surrounded by a broken line BK. In FIG. 5A, the image CL of clutter is not rendered in the blood flow image CD, and the visibility of the image is improved.

The ultrasound diagnosis apparatus 1 of the embodiment includes the tomographic image data generator 13, the blood flow information generator 14, the change detector 15, and the condition changing unit 16. In response to ultrasound waves transmitted to a subject, the tomographic image data generator 13 receives a reception signal based on reflected waves from the subject, and sequentially obtains tomographic image data of the subject for a plurality of times. The blood flow information generator 14 receives the reception signal, and performs processing on the reception signal based on the signal processing conditions including a velocity threshold related to blood flow information of the subject to obtain the blood flow information. The change detector 15 receives a plurality of pieces of tomographic image data for different times, and detects the magnitude of change in the time axis direction of the pieces of tomographic image data. The condition changing unit 16 changes the signal processing conditions based on the magnitude of change in the time axis direction of tomographic image data detected by the change detector 15. When the condition changing unit 16 changes the signal processing conditions, the blood flow information generator 14 performs processing on the reception signal based on changed signal processing conditions to obtain the blood flow information. In this manner, by detecting a time-series variation in tomographic image data, the signal processing conditions for the blood flow information are changed based on the magnitude of the variation. Thereby, the blood flow information generator 14 can obtain blood flow information while reducing clutter and motion artifacts based on the signal processing conditions corresponding to the amplitude of the movement of subject tissue or the ultrasound probe 10. Thus, the reduction of clutter and motion artifacts can be achieved.

### <First Modification>

### [Configuration]

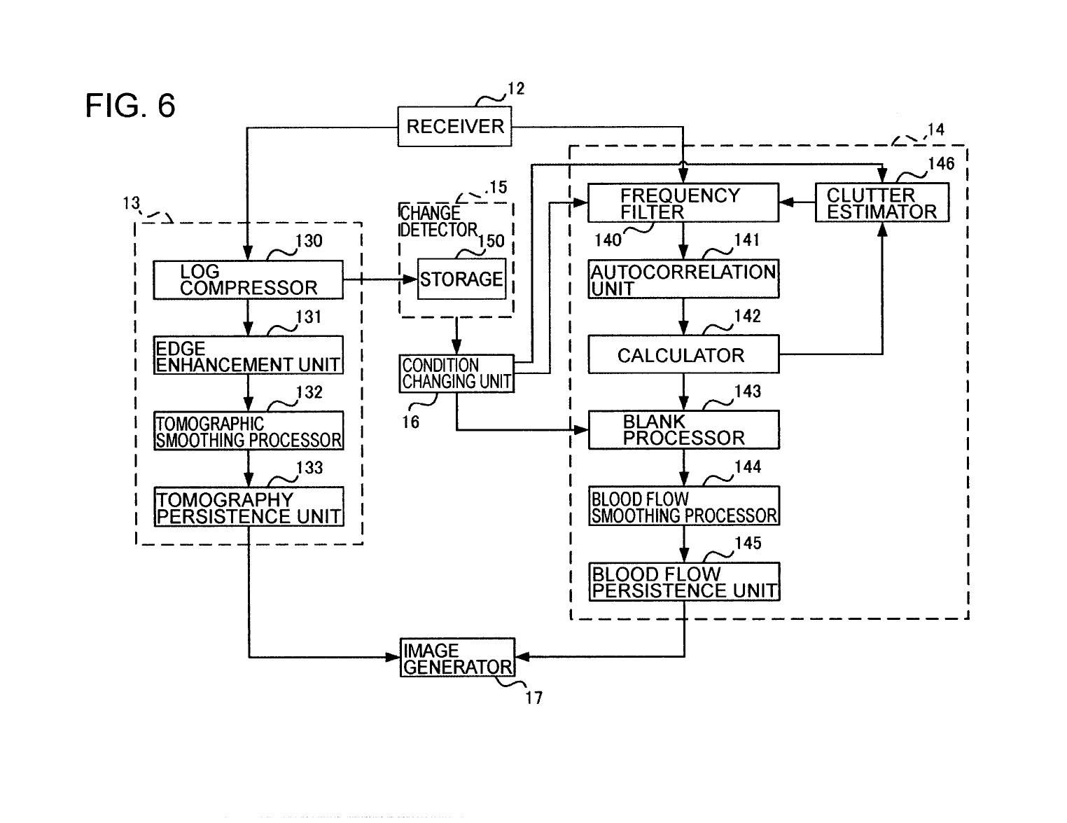

FIG. 6 is a block diagram of the ultrasound diagnosis apparatus 1 of this modification. The ultrasound diagnosis apparatus 1 of this modification is different from that of the above embodiment in mostly the configuration of the blood flow information generator 14. Incidentally, in this modification, like parts as in the above embodiment are designated by like reference numerals, and the description thereof is not repeated. The differences are mainly described below.

The blood flow information generator 14 includes a clutter estimator 146, in addition to the frequency filter 140, the autocorrelation unit 141, the calculator 142, the blank processor 143, the blood flow smoothing processor 144, and the blood flow persistence unit 145.

The clutter estimator 146 estimates clutter contained in the blood flow information. In other words, the clutter estimator 146 estimates how much clutter is present in a signal that has passed through the frequency filter 140 based on the velocity, variance, and power of the blood flow calculated by the calculator 142. For example, the clutter estimator 146 stores a reference value as a signal processing condition with respect to each of the velocity, variance, and power of the blood flow. The clutter estimator 146 compares the calculated value of each of the velocity, variance, and power of the blood flow received from the calculator 142 with the reference value. The larger the difference between the calculated value and the reference value is, the more clutter the clutter estimator 146 estimates. Incidentally, the clutter estimator 146 stores a function or table data representing the correlation between the difference and the amount of clutter, and estimates the amount of clutter by performing operation with reference to the table data or using the function. For example, the filter characteristics of the frequency filter 140 are changed such that the passband becomes narrower as clutter estimated by the clutter estimator 146 increases.

The condition changing unit 16 receives the magnitude of change in the time axis direction of tomographic image data detected by the change detector 15, and changes the reference value as a signal processing condition in the clutter estimator 146 based on the magnitude of change. As the change becomes greater in the time axis direction of tomographic image data, the condition changing unit 16 changes the reference value of the clutter estimator 146 such that the clutter estimator 146 estimates more clutter.

### [Operation]

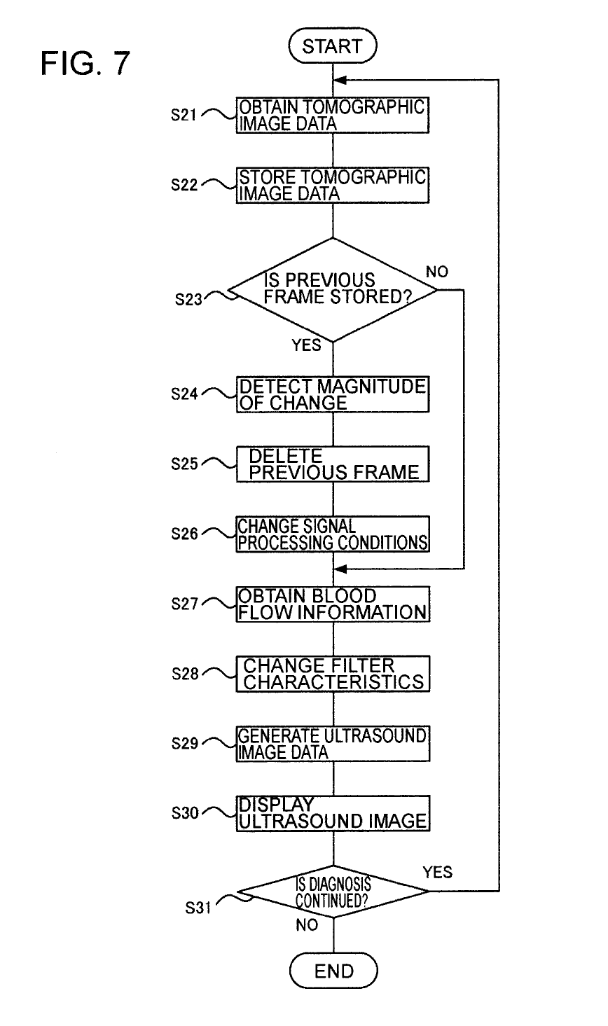

FIG. 7 is a flowchart illustrating the operation of the ultrasound diagnosis apparatus 1 of this modification.

### (S21)

In response to ultrasound waves transmitted to a subject, the tomographic image data generator 13 receives, from the receiver 12, a reception signal based on reflected waves from the subject, and obtains tomographic image data of the subject. At this time, the log compressor 130 performs compression by logarithmic conversion on the reception signal, and outputs it to the edge enhancement unit 131 and the storage 150. The edge enhancement unit 131 emphasizes the edges of the signal received from the log compressor 130, and outputs it to the tomographic smoothing processor 132. The tomographic smoothing processor 132 performs smoothing filtering on the signal received from the edge enhancement unit 131, and outputs it to the tomography persistence unit 133. The tomography persistence unit 133 performs weighted addition of the signal newly received from the tomographic smoothing processor 132 to signals received therefrom in the past. The tomography persistence unit 133 outputs the resultant signal to the image generator 17.

### (S22)

The storage 150 stores the tomographic image data received from the log compressor 130.

### (S23)

Besides the latest tomographic image data (latest frame) received from the log compressor 130, if the storage 150 stores previous tomographic image data (previous frame) obtained previous to the latest data (YES in step S23), the process proceeds to step S24. When the storage 150 does not store the previous tomographic image data (previous frame) as well as the latest tomographic image data (latest frame) received from the log compressor 130 (NO in step S23), the process proceeds to step S27.

### (S24)

The change detector 15 retrieves, from the storage 150, the latest frame as tomographic image data obtained as to the most recent time, and a previous frame as previous tomographic image data obtained as to the time before the most recent time. The change detector 15 detects the magnitude of change in the time axis direction of tomographic image data based on the tomographic image data retrieved. The change detector 15 outputs the magnitude of change thus detected to the condition changing unit 16.

### (S25)

The storage 150 deletes the previous frame stored therein, and a frame stored as the latest frame becomes a previous frame.

### (S26)

The condition changing unit 16 changes the signal processing conditions of the blood flow information generator 14 based on the magnitude of change in the time axis direction of tomographic image data detected by the change detector 15. The signal processing conditions includes the frequency characteristics of the frequency filter 140, the velocity threshold, the variance threshold, or the lower power threshold and the upper power threshold of the blank processor 143, or the combination thereof. In this case, the greater the change the change detector 15 has detected, the condition changing unit 16 raises the velocity threshold, reduces the variance threshold, increases the lower power threshold, and lowers the dupper power threshold. In addition, having received the magnitude of change in the time axis direction of tomographic image data detected by the change detector 15, the condition changing unit 16 changes the reference value as a signal processing condition in the clutter estimator 146 based on the magnitude of change.

### (S27)

Upon receipt of the reception signal from the receiver 12, the blood flow information generator 14 performs processing on the reception signal based on the signal processing conditions to obtain blood flow information of the subject. At this time, the frequency filter 140 performs filtering based on the frequency characteristics on the reception signal received from the receiver 12. The autocorrelation unit 141 performs an autocorrelation calculation with respect to the signal received from the frequency filter 140. The calculator 142 calculates the velocity, variance, or power of the blood flow in the subject, or a combination of these. Of velocities received from the calculator 142, the blank processor 143 outputs a velocity equal to or above the velocity threshold to the blood flow smoothing processor 144. Further, of variances received from the calculator 142, the blank processor 143 outputs a variance equal to or above the variance threshold to the blood flow smoothing processor 144. Still further, of powers received from the calculator 142, the blank processor 143 outputs a power equal to or above the lower power threshold as well as equal to or below the upper power threshold to the blood flow smoothing processor 144. The blood flow smoothing processor 144 performs smoothing filtering on the output from the blank processor 143, and outputs it to the blood flow persistence unit 145. The blood flow persistence unit 145 performs weighted addition of the signal newly received from the blood flow smoothing processor 144 to signals received therefrom in the past. The blood flow persistence unit 145 outputs the signal obtained by the weighted addition to the image generator 17.

### (S28)

The clutter estimator 146 estimates how much clutter is present in a signal that has passed through the frequency filter 140 based on the velocity, variance, and power of the blood flow calculated by the calculator 142, and changes the filter characteristics of the frequency filter 140.

### (S29)

The image generator 17 generates ultrasound image data based on the tomographic image data output from the tomographic image data generator 13 and the blood flow information output from the blood flow information generator 14.

### (S30)

The display controller 18 receives the ultrasound image data from the image generator 17, and displays an ultrasound image on the display 19 based on the ultrasound image data.

### (S31)

When the ultrasound diagnosis is continued (YES in step S31), the process returns to the step S21. If not (NO in step S31), the operation ends.

The ultrasound diagnosis apparatus 1 of the modification includes the tomographic image data generator 13, the blood flow information generator 14, the change detector 15, and the condition changing unit 16. In response to ultrasound waves transmitted to a subject, the tomographic image data generator 13 receives a reception signal based on reflected waves from the subject, and sequentially obtains tomographic image data of the subject for a plurality of times. The blood flow information generator 14 receives the reception signal, and performs processing on the reception signal based on the signal processing conditions including a velocity threshold related to blood flow information of the subject to obtain the blood flow information. The change detector 15 receives a plurality of pieces of tomographic image data for different times, and detects the magnitude of change in the time axis direction of the pieces of tomographic image data. The condition changing unit 16 changes the signal processing conditions based on the magnitude of change in the time axis direction of tomographic image data detected by the change detector 15. When the condition changing unit 16 changes the signal processing conditions, the blood flow information generator 14 performs processing on the reception signal based on changed signal processing conditions to obtain the blood flow information. The blood flow information generator 14 further includes the clutter estimator 146. The clutter estimator 146 estimates how much clutter is present in a signal that has passed through the frequency filter 140 based on the velocity, variance, and power of the blood flow calculated by the calculator 142. Having received the magnitude of change in the time axis direction of tomographic image data detected by the change detector 15, the condition changing unit 16 changes the reference value as a signal processing condition in the clutter estimator 146 based on the magnitude of change. In this manner, the ultrasound diagnosis apparatus 1 determines a reference value for estimating clutter contained in a signal that has passed through the frequency filter 140 according to the magnitude of change in the time axis direction of tomographic image data, and changes the filter characteristics of the frequency filter 140 based on the clutter estimated. Thereby, the ultrasound diagnosis apparatus 1 can sequentially estimate the amount of clutter according to the amplitude of the movement of subject tissue, and provide a feedback to the frequency filter 140. Thus, the reduction of clutter and motion artifacts can be achieved.

### <Second Modification>

The ultrasound diagnosis apparatus 1 of this modification is capable of generating an ultrasound image in which an image based on clutter and motion artifacts can be illustrated weaker than an image representing blood flow information. The ultrasound diagnosis apparatus 1 of this modification is different from that of the above embodiment in mostly the configuration of the blood flow information generator 14. Incidentally, in this modification, like parts as in the above embodiment are designated by like reference numerals, and the description thereof is not repeated. The differences are mainly described below.

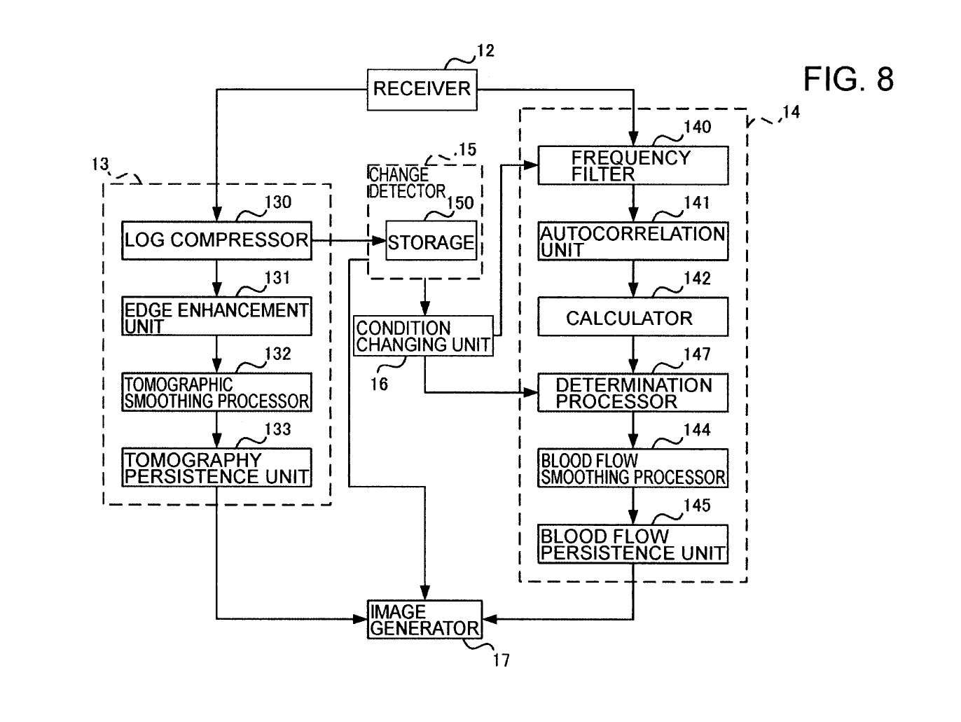

FIG. 8 is a block diagram illustrating a configuration of the blood flow information generator 14 of this modification. The blood flow information generator 14 determines whether a reception signal represents blood flow information based on the signal processing conditions. The blood flow information generator 14 includes a determination processor 147 in place of the blank processor 143. The determination processor 147 determines whether the blood flow information calculated by the calculator 142 is information representing the hemodynamics of the subject.

The determination processor 147 stores a velocity threshold. The determination processor 147 compares a velocity received from the calculator 142 with the velocity threshold to determine whether the velocity is equal to or higher than the velocity threshold, or less than the velocity threshold. The velocity equal to or higher than the velocity threshold corresponds to the information representing the hemodynamics of the subject. The determination processor 147 outputs velocity information as to the velocity together with supplementary information indicating the determination result to the blood flow smoothing processor 144. At this time, even if the velocity is less than the velocity threshold, the determination processor 147 does not remove the velocity, and outputs the velocity information together with supplementary information indicating the determination result to the blood flow smoothing processor 144. Incidentally, when the condition changing unit 16 changes the velocity threshold, the determination processor 147 makes this determination with reference to a new velocity threshold.

The determination processor 147 also stores a variance threshold. The determination processor 147 compares a variance received from the calculator 142 with the variance threshold to determine whether the variance is equal to or more than the variance threshold, or less than the variance threshold. The variance equal to or more than the variance threshold corresponds to the information representing the hemodynamics of the subject. The determination processor 147 outputs the variance together with supplementary information indicating the determination result to the blood flow smoothing processor 144. At this time, even if the variance is less than the variance threshold, the determination processor 147 does not remove the variance, and outputs the variance together with supplementary information indicating the determination result to the blood flow smoothing processor 144. Incidentally, when the condition changing unit 16 changes the variance threshold, the determination processor 147 makes this determination with reference to a new variance threshold.

In addition, the determination processor 147 stores a lower power threshold and an upper power threshold. The determination processor 147 compares a power received from the calculator 142 with the lower power threshold and the upper power threshold to determine whether the power is equal to or more than the lower power threshold, or equal to or less than the upper power threshold. The power equal to or more than the lower power threshold as well as equal to or less than the upper power threshold corresponds to the information representing the hemodynamics of the subject. The determination processor 147 outputs the power together with supplementary information indicating the determination result to the blood flow smoothing processor 144. At this time, even if the power is below the lower power threshold and above the upper power threshold, the determination processor 147 does not remove the power, and outputs the power together with supplementary information indicating the determination result to the blood flow smoothing processor 144. Incidentally, when the condition changing unit 16 changes the lower power threshold and the upper power threshold, the determination processor 147 makes this determination with reference to new thresholds.

Through the various determination processes by the determination processor 147, the image generator 17 is fed with blood flow information (color Doppler information) including supplementary information that represents various determination results.

The change detector 15 outputs the magnitude of change detected to the condition changing unit 16 and also to the image generator 17. The image generator 17 generates ultrasound image data by which an image based on a reception signal determined as not representing blood flow information can be displayed weaker than an image based on a reception signal determined as representing blood flow information based on the determination result by the determination processor 147. The image generator 17 generates ultrasound image data based on the tomographic image data obtained by the tomographic image data generator 13, the blood flow information obtained by the blood flow information generator 14, and the magnitude of change obtained by the change detector 15. The image generator 17 generates ultrasound image data in which blood flow information determined as representing the hemodynamics of the subject and that determined as not representing the hemodynamics are rendered in different modes.

For example, the image generator 17 identifies the determination result obtained by the determination processor 147 with reference to supplementary information of the blood flow information. The image generator 17 generates ultrasound image data such that the brightness of the pixels of blood flow information determined as not representing the hemodynamics of the subject is less than the brightness of the pixels of blood flow information determined as representing the hemodynamics.

At this time, the greater the magnitude of change the change detector 15 has detected, the lower the brightness the image generator 17 sets for pixels of blood flow information determined as not representing the hemodynamics of the subject. The decrease in the brightness corresponds to reducing the display power for an image based on clutter and motion artifacts. For example, depending on the magnitude of change obtained by the change detector 15, the image generator 17 reduces the brightness of the pixels of blood flow information determined as not representing the hemodynamics of the subject in a stepwise fashion. Note that, depending on the magnitude of change obtained by the change detector 15, the image generator 17 may reduce the brightness of the pixels of blood flow information determined as not representing the hemodynamics of the subject in a non-stepwise fashion.

The image generator 17 outputs the ultrasound image data thus generated to the display controller 18. The display controller 18 displays an ultrasound image on the display 19 based on the ultrasound image data. With this, as the magnitude of change detected by the change detector 15 increases, the pixels of blood flow information determined as not representing the hemodynamics of the subject are displayed in a darker color as compared to the pixels of blood flow information determined as representing the hemodynamics. Therefore, while viewing the ultrasound image, the user can recognize a portion of the pixels displayed in a dark color as a part where clutter and motion artifacts are rendered.

The image generator 17 may generate ultrasound image data so that the pixels of blood flow information determined as not representing the hemodynamics of the subject are displayed as a watermark. For example, the image generator 17 adds up Red-Green-Blue (RGB) signals of the pixels of blood flow information determined as not representing the hemodynamics of the subject and the pixels of tomographic image data in the same coordinates as the pixels of the blood flow information.

In this case, as the magnitude of change detected by the change detector 15 increases, the image generator 17 reduces the addition ratio of RGB signals of the pixels of blood flow information determined as not representing the hemodynamics of the subject as well as increasing that of RGB signals of the pixels of tomographic image data in the same coordinates as the pixels of the blood flow information. This reduction and increase of the addition ratio of RGB signals corresponds to reducing the display power for an image based on clutter and motion artifacts. For example, depending on the magnitude of change obtained by the change detector 15, the image generator 17 reduces the addition ratio of RGB signals of the pixels of blood flow information determined as not representing the hemodynamics of the subject as well as increasing that of RGB signals of the pixels of tomographic image data in the same coordinates as the pixels of the blood flow information in a stepwise fashion. Note that, depending on the magnitude of change obtained by the change detector 15, the image generator 17 may reduce the addition ratio of RGB signals of the pixels of blood flow information determined as not representing the hemodynamics of the subject as well as increasing that of RGB signals of the pixels of tomographic image data in the same coordinates as the pixels of the blood flow information in a non-stepwise fashion. The image generator 17 does not perform this addition for the pixels of blood flow information determined as representing the hemodynamics of the subject. The image generator 17 generates ultrasound image data for the pixels of blood flow information determined as representing the hemodynamics of the subject such that a Doppler image is superimposed on a tomographic image.

In the display of an ultrasound image based on the ultrasound image data thus generated, with respect to a portion of the pixels of blood flow information determined as not representing the hemodynamics of the subject, as the magnitude of change detected by the change detector 15 increases, RGB signals of the pixels of the tomographic image are displayed at a high ratio, while RGB signals of the color Doppler image is displayed at a low ratio as a watermark. A portion of the pixels of blood flow information determined as representing the hemodynamics of the subject has no watermark display, and the color Doppler image is superimposed on the tomographic image. Therefore, while viewing the ultrasound image, the user can recognize a portion of the pixels with a watermark of the color Doppler image as a part where clutter and motion artifacts are rendered.

The ultrasound image processing method of the above embodiments may be implemented by computer programs. Such computer programs can be stored in an arbitrary recording medium that is readable by a computer. Examples of the recording medium include, for example, a semiconductor memory, an optical disk, a magneto-optical disk, a magnetic storage medium, and the like. The computer programs may be transmitted and received through a network such as the Internet or LAN.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus, comprising processing circuitry configured to:
receive a reception signal based on reflected waves returned from a subject in response to ultrasound waves transmitted to the subject, and sequentially obtain tomographic image data of the subject for a plurality of times;
perform processing on the reception signal based on signal processing conditions including a velocity threshold related to blood flow information of the subject to obtain the blood flow information;
detect a magnitude of change in time axis direction of a plurality of pieces of tomographic image data for different times;
change the signal processing conditions based on the magnitude of change in the time axis direction of the tomographic image data; and
when having changed the signal processing conditions, perform the processing on the reception signal based on changed signal processing conditions to obtain the blood flow information.

2. The ultrasound diagnosis apparatus of claim 1, wherein the processing circuitry is further configured to
calculate a velocity of blood flow in the subject,
obtain the velocity as the blood flow information when the velocity is equal to or above the velocity threshold, which is a threshold value to obtain the velocity as the blood flow information,
change the velocity threshold to a new velocity threshold, and
after having changed the velocity threshold, obtain a velocity as the blood flow information when the velocity is equal to or above the new velocity threshold.

3. The ultrasound diagnosis apparatus of claim 2, wherein the processing circuitry is further configured to increase the velocity threshold when detecting a greater magnitude of change in the time axis direction.

4. The ultrasound diagnosis apparatus of claim 1, wherein the processing circuitry is further configured to
calculate a variance of blood flow in the subject, wherein the signal processing conditions include a variance threshold, which is a threshold value to obtain the variance as the blood flow information,
obtain the variance as the blood flow information when the variance is equal to or above the variance threshold,
change the variance threshold to a new variance threshold, and
after having changed the variance threshold, obtain a variance as the blood flow information when the variance is equal to or above the new variance threshold.

5. The ultrasound diagnosis apparatus of claim 4, wherein the processing circuitry is further configured to reduce the variance threshold when detecting a greater magnitude of change in the time axis direction.

6. The ultrasound diagnosis apparatus of claim 1, wherein the processing circuitry is further configured to
calculate a power of blood flow in the subject, wherein the signal processing conditions include a lower power threshold and an upper power threshold, which are threshold values to obtain the power as the blood flow information,
obtain the power as the blood flow information when the power is equal to or above the lower power threshold and equal to or below the upper power threshold,
change the lower power threshold and the upper power threshold to a new lower power threshold and a new upper power threshold, respectively, and
after having changed the lower power threshold and the upper power threshold, obtain a power as the blood flow information when the power is equal to or above the new lower power threshold and equal to or below the new upper power threshold.

7. The ultrasound diagnosis apparatus of claim 6, wherein the processing circuitry is further configured to increase the lower power threshold and reduce the upper power threshold when detecting a greater magnitude of change in the time axis direction.

8. The ultrasound diagnosis apparatus of claim 1, further comprising a frequency filter configured to perform filtering on the reception signal based on specified frequency characteristics, wherein
the signal processing conditions include frequency characteristics of the frequency filter, and
the processing circuitry is further configured to
change the frequency characteristics of the signal processing conditions, and
when having changed the frequency characteristics, perform the processing on the reception signal based on changed frequency characteristics to obtain the blood flow information.

9. The ultrasound diagnosis apparatus of claim 8, wherein the processing circuitry is further configured to
estimate clutter contained in the blood flow information based on a reference value stored in advance and the blood flow information to change the frequency characteristics based on the clutter, and
change the reference value based on the magnitude of change in the time axis direction.

10. The ultrasound diagnosis apparatus of claim 1, wherein the processing circuitry is further configured to
determine whether the reception signal represents the blood flow information based on the signal processing conditions, and
generate, based on a determination result, ultrasound image data by which an image based on the reception signal determined as not representing the blood flow information can be displayed weaker than an image based on the reception signal determined as representing the blood flow information.

11. An ultrasound image processing method for processing a reception signal based on reflected waves returned from a subject in response to ultrasound waves transmitted to the subject, the method comprising:
sequentially obtaining tomographic image data of the subject for a plurality of times;
performing processing on the reception signal based on signal processing conditions including a velocity threshold related to blood flow information of the subject to obtain the blood flow information;
detecting a magnitude of change in time axis direction of a plurality of pieces of tomographic image data for different times; and
when having changed the signal processing conditions based on the magnitude of change in the time axis direction, obtaining the blood flow information based on changed signal processing conditions.
